(19) United States
(12) Patent Application Publication
(10) Pub. No.: US 2019/0246988 A1
(43) Pub. Date: Aug. 15, 2019

(54) PHYSIOLOGICAL SIGNAL CORRECTION DEVICE, CORRECTION METHOD, AND WEARABLE DEVICE WITH CORRECTION FUNCTION

(71) Applicant: Industrial Technology Research Institute, Hsinchu (TW)

(72) Inventors: Ming-Huan Yang, Hsinchu City (TW); Cheng-Chung Lee, Hsinchu City (TW); Shih-Kuang Chiu, Taichung City (TW); Kuang-Ching Fan, Hsinchu County (TW)

(73) Assignee: Industrial Technology Research Institute, Hsinchu (TW)

(21) Appl. No.: 16/262,945

(22) Filed: Jan. 31, 2019

Related U.S. Application Data
(60) Provisional application No. 62/629,130, filed on Feb. 12, 2018.

(30) Foreign Application Priority Data
Aug. 24, 2018 (TW) 107129561

(51) Int. Cl. A61B 5/00; A61B 5/0205; A61B 5/0424; A61B 5/024

(52) U.S. Cl. CPC A61B 5/721; A61B 5/02055; A61B 5/0424; A61B 5/02438; A61B 5/04004; A61B 5/688; A61B 5/02405; A61B 5/0002; A61B 5/743; A61B 5/6833

(57) ABSTRACT

A physiological signal correction device, a correction method, and a wearable device with a correction function are provided. The physiological signal correction device includes a physiological signal sensor, a warping sensor, and a signal processing device. The physiological signal sensor is attached to an object to be detected to obtain a physiological signal value from at least one sensing electrode. The warping sensor is disposed on the physiological signal sensor and detects whether a warping condition of the physiological signal sensor with respect to the object to be detected occurs. The signal processing device corrects the physiological signal value provided by the physiological signal sensor according to the warping condition. The warping condition is caused by a distance between a part of the sensing electrode and the object to be detected or a change in a contact area between a part of the sensing electrode and the object to be detected.

### CROSS-REFERENCE TO RELATED APPLICATION

This application claims the priority benefit of U.S. provisional application Ser. No. 62/629,130, filed on Feb. 12, 2018, and Taiwan application serial no. 107129561, filed on Aug. 24, 2018. The entirety of each of the above-mentioned patent application is hereby incorporated by reference herein and made a part of this specification.

### BACKGROUND

### Technical Field

The disclosure relates to a signal detection and processing technique and relates to a physiological signal correction device, a correction method for a physiological signal, and a wearable device with a physiological signal correction function.

### Description of Related Art

In wearable biomedical detection techniques, a physiological signal detection device (e.g., a sensing electrode patch or a sensor) put on the body, and the physiological signals of the wearer may be recorded at any time in a non-invasive manner to thereby detect the body physiological status (e.g., a body temperature, a pulse, a heart rate, a respiratory rate, etc.) of the wearer. Moreover, the device can also send an alert to remind the wearer, or even achieve the effect of promptly sending an alert and seeking help when symptoms occur. Therefore, with advance wearable biomedical detection techniques, significant convenience has been created for wearers such as patients convalescing at home, patients with a clinical history of heart disease, or elderly people living alone.

However, there is still room for improvement in user experience due to the existing sensing electrode patch that has to be closely attached to the wearer's skin is often warped or falls off. Specifically, a general physiological signal detection device (e.g., a sensing electrode patch or a sensor) has to be closely attached to the wearer's skin in order to obtain accurate physiological signals. The detected physiological signal is distorted due to tension caused by sweat or exercise causing some or all of sensing electrode(s) patch to fall off or not adhere to the skin. In the related art, the solutions generally involve enhancement of the adhesiveness of the sensual electrode patch to enhance the adhesion to the skin. However, in such solutions, the wearer is generally more uncomfortable, fall-off is still possible, or the arrangement of the sensing electrode patch is more inconvenient. Moreover, in many cases, the wearer is not aware that the sensing electrode patch has fallen off and the physiological signal is distorted, which results in poor accuracy of the physiological signal detection.

### SUMMARY

The physiological signal correction device of the embodiments of the disclosure includes a physiological signal sensor, a warping sensor, and a signal processing device. The physiological signal sensor includes at least one sensing electrode. The physiological signal sensor is attached to an object to be detected to obtain a physiological signal value from the sensing electrode. The warping sensor is disposed on the physiological signal sensor. The warping sensor detects whether a warping condition of the physiological signal sensor with respect to the object to be detected occurs. The signal processing device is coupled to the physiological signal sensor and the warping sensor. The signal processing device corrects the physiological signal value provided by the physiological signal sensor according to the warping condition provided by the warping sensor, wherein the warping condition is caused by a distance between a part of the sensing electrode and the object to be detected or a change in a contact area between a part of the sensing electrode and the object to be detected.

The correction method for a physiological signal of the embodiments of the disclosure is adapted for a physiological signal correction device including a physiological signal sensor and a warping sensor. The warping sensor is disposed on the physiological signal sensor. The correction method includes steps below. A physiological signal value is obtained from the physiological signal sensor when the physiological signal sensor is attached to an object to be detected. The warping sensor detects whether a warping condition of the physiological signal sensor with respect to the object to be detected occurs, wherein the warping condition is caused by a distance between a part of the physiological signal sensor and the object to be detected or a change in a contact area between a part of at least one sensing electrode and the object to be detected. The physiological signal value provided by the physiological signal sensor is corrected according to the warping condition provided by the warping sensor.

The wearable device with a correction function of the embodiments of the disclosure includes a physiological signal sensor, a warping sensor, and a signal processing device. The physiological signal sensor includes at least one sensing electrode. The physiological signal sensor is attached to an object to be detected to obtain a physiological signal value from the sensing electrode. The warping sensor is disposed on the physiological signal sensor. The warping sensor detects whether a warping condition of the physiological signal sensor with respect to the object to be detected occurs. The signal processing device is coupled to the physiological signal sensor and the warping sensor. The signal processing device corrects the physiological signal value provided by the physiological signal sensor according to the warping condition provided by the warping sensor, wherein the warping condition is caused by a distance between a part of the sensing electrode and the object to be detected or a change in a contact area between a part of the sensing electrode and the object to be detected.

To provide a further understanding of the aforementioned and other content of the disclosure, exemplary embodiments, together with the reference drawings, are described in detail below.

### BRIEF DESCRIPTION OF THE DRAWINGS

The accompanying drawings are included to provide a further understanding of the disclosure and are incorporated in and constitute a part of this specification. The drawings illustrate exemplary embodiments of the disclosure and, together with the description, serve to explain the principles of the disclosure.

FIG. 1 is a block diagram illustrating a physiological signal correction device according to a first embodiment of the disclosure.

FIG. 2A to FIG. 2C are diagrams illustrating corresponding positional relationships of a physiological signal sensor and a warping sensor.

FIG. 2D to FIG. 2E are schematic diagrams illustrating a sensing electrode patch composed of the warping sensor and the physiological signal sensor and an object to be detected.

FIG. 3 is a schematic diagram illustrating an implementation of the warping sensor of the sensing electrode patch in FIG. 2D.

FIG. 4A and FIG. 4B are schematic diagrams illustrating integrated structures of the physiological signal sensor and the warping sensor.

FIG. 5A to FIG. 5F are diagrams illustrating corresponding positional relationships of the physiological signal sensor, the warping sensor, and a signal processing device in FIG. 1.

FIG. 6A and FIG. 6B are schematic diagrams illustrating an implementation of the warping sensor as a photosensitive-type sensor.

FIG. 7 is a block diagram illustrating a physiological signal correction device, an information displaying device, and a cloud server according to a second embodiment of the disclosure.

FIG. 8 is a circuit block diagram illustrating a physiological signal correction device according to a third embodiment of the disclosure.

FIG. 9 is a flowchart illustrating a correction method for a physiological signal according to an embodiment of the disclosure.

### DESCRIPTION OF THE EMBODIMENTS

In order to make the disclosure more comprehensible, embodiments are described below as the examples to prove that the disclosure can actually be realized. In addition, wherever possible, elements/components/steps denoted by the same reference numerals in drawings and embodiments represent the same or similar parts.

Embodiments of the disclosure provide a physiological signal correction device, a correction method for a physiological signal, and a wearable device with a physiological signal correction function that can detect and feedback a warping condition of mutual detachment between at least one sensing electrode and an object to be detected (e.g., a user's skin) and compensate and correct the physiological signal according to the warping condition. Thereby, the physiological signal detected in the embodiments of the disclosure can exhibit high accuracy.

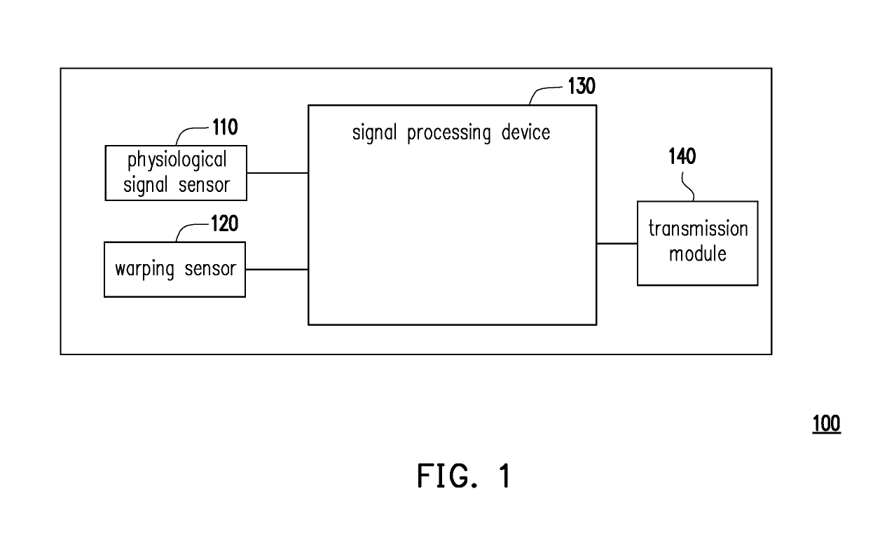

FIG. 1 is a block diagram illustrating a physiological signal correction device 100 according to a first embodiment of the disclosure. The physiological signal correction device 100 may be a wearable device with a physiological signal correction function. The physiological signal correction device 100 mainly includes a physiological signal sensor 110, a warping sensor 120, and a signal processing device 130. The entire physiological signal correction device 100 may be implemented in the form of a physiological signal sensing patch.

The physiological signal sensor 110 includes one or more sensing electrodes. The physiological signal sensor 110 is attached to an object to be detected to obtain a physiological signal value from the sensing electrode(s). In the embodiment, the “physiological signal” may be a body temperature, a pulse, a heart rate, a respiratory rate, an electroencephalography (EEG), an electromyography (EMG), an electroneurogram (ENG), an electroretinogram (ERG), an electrogastrogram (EGG), an electroneuromyography (ENMG), an electrocorticography (ECoG), an electrooculogram (EOG), an electronystagmography (ENG), a nystagmus electrical signal (ENG), etc., and the detection type of the physiological signal sensor 110 for the physiological signal is determined by the use and requirements of the physiological signal correction device 100. In the embodiment, the “physiological signal value” is the value of the physiological signal of the types above. In the embodiment, the “object to be detected” is mainly the skin of a user (or referred to as a wearer, e.g., a person or an animal), and a person implementing the embodiment may also regard another object as the object to be detected as long as the physiological signal value can be sensed from the object to be detected. The warping sensor 120 is disposed on the physiological signal sensor 110. The physiological signal sensor 110 and the warping sensor 120 may be made of a conformal or flexible material.

The warping sensor 120 is mainly used to detect whether a warping condition of the physiological signal sensor 110 with respect to the object to be detected occurs. The signal processing device 130 is coupled to the physiological signal sensor 110 and the warping sensor 120. The signal processing device 130 corrects the physiological signal value provided by the physiological signal sensor 110 according to the warping condition provided by the warping sensor 120. The physiological signal correction device 100 further includes a transmission module 140. The transmission module 140 is coupled to the signal processing device 130. The physiological signal correction device 100 may utilize the transmission module 140 to transmit the detected and corrected physiological signal value to an external information displaying device. Accordingly, in the embodiment, the physiological signal correction device 100 can detect and correct the physiological signal value in real time and transmit the physiological signal value to the external information displaying device.

The “warping condition” described in the embodiment is caused by the distance between a part of the sensing electrode(s) on the physiological signal sensor 110 and the object to be detected or a change in the contact area between a part of the sensing electrode(s) and the object to be detected. For example, the “warping condition” may include two conditions. The first condition occurs when the distance between the sensing electrode(s) on a part of the physiological signal sensor 110 or the partial physiological signal sensor 110 and the object to be detected is too far, such that the physiological signal sensor 110 cannot detect the physiological signal. In this warping condition, a warping value may be expressed in an area percentage of mutual attachment and detachment between the physiological signal sensor 110 and the object to be detected. The other condition occurs when the physiological signal sensor 110 and the object to be detected are indeed closely attached to each other, but, due to deformation and/or creases of the physiological signal sensor 110, a part of the sensing electrode(s) cannot function normally as a result of a change in the contact area with the object to be detected. In this warping condition, the warping value may be expressed in an area percentage of the area where the physiological signal sensor 110 is attached to the object to be detected and is deformed. For example, the physiological signal sensor 110 is originally closely attached to the object to be detected, but a part of the physiological signal sensor 110 falls off the wearer's skin due to sweat or the movement of the wearer, such that the detected physiological signal is distorted. Alternatively, the physiological signal sensor 110 is significantly deformed or creased along with the wearer's skin, such that the detected physiological signal is distorted. The physiological signal sensor 110 may include one or multiple types of warping sensors 120 to more accurately detect the warping condition above.

In the related art, the distorted physiological signal detected in the condition above cannot correctly reflect the real physiological state of the wearer, which thus causes the wearable device to be unable to function normally. Only after the physiological signal sensor 110 is reattached closely to the skin can the wearable device exert its proper function again. In comparison, in the embodiment of the disclosure, the warping sensor 120 is used to obtain the warping value associated with the “warping condition”, and the warping value is used to query the correction database located in the physiological signal correction device 100 to generate the corrected physiological signal value, which thereby compensates or corrects the value generated from the physiological signal sensor 110 and prolongs the time during which the wearable device can function normally in the case of a slight warping condition.

In the embodiment, the area percentage of mutual attachment between the physiological signal sensor 110 and the object to be detected (the wearer's skin) may be used as the warping value of the “warping condition”. In other words, the higher the area percentage of mutual attachment between the physiological signal sensor 110 and the object to be detected, the lower the degree to which the physiological signal sensor 110 is detached from the skin and thus the less the physiological signal value that needs to be compensated or corrected. The lower the area percentage of mutual attachment between the physiological signal sensor 110 and the object to be detected, the higher the degree to which the physiological signal sensor 110 is detached from the skin and thus the more the physiological signal value that needs to be compensated or corrected.

It is noted that, when the physiological signal sensor 110 is unable to obtain the physiological signal value or the physiological signal value has fallen below a predetermined value, the physiological signal correction device 100 does not compensate or correct the physiological signal value by using the warping value corresponding to the warping condition. Instead, the physiological signal correction device 100 notifies the wearer or the person maintaining the physiological signal correction device 100 by other means to alert that the physiological signal correction device 100 at this time cannot exert its proper function.

A person implementing the embodiment may adjust the correspondence relationship between the physiological signal sensor 110 and the warping sensor 120 according to the requirements, which is described in the following examples and drawings. If the detection method of the warping sensor 120 is distinguished, the types of the warping sensor 120 may include a photosensitive-type sensor (change in a photocurrent), a vibration-type sensor (sensing a change in the vibration frequency on the skin), a resistance-type sensor (change in the resistance value on the skin surface), a capacitance-type sensor (change in the capacitance value on the skin surface), a microwave-type sensor (detecting a change in the distance between the sensor and the skin by using microwave techniques), or a combination of the various sensors above. If the warping sensor 120 is placed at a position of the physiological signal sensor 110 is distinguished, the warping sensor 120 may be an entire surface-type, regional-type, or array-type sensor.

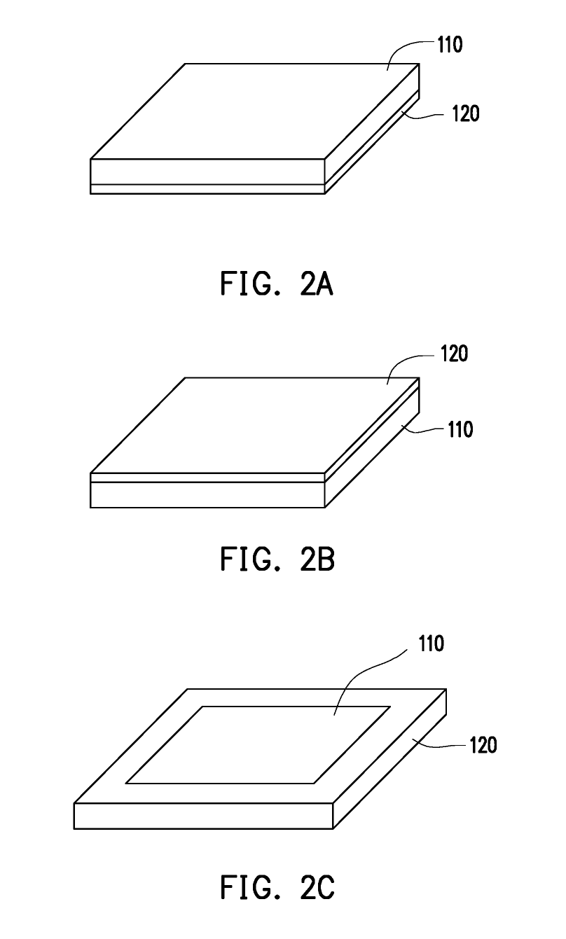

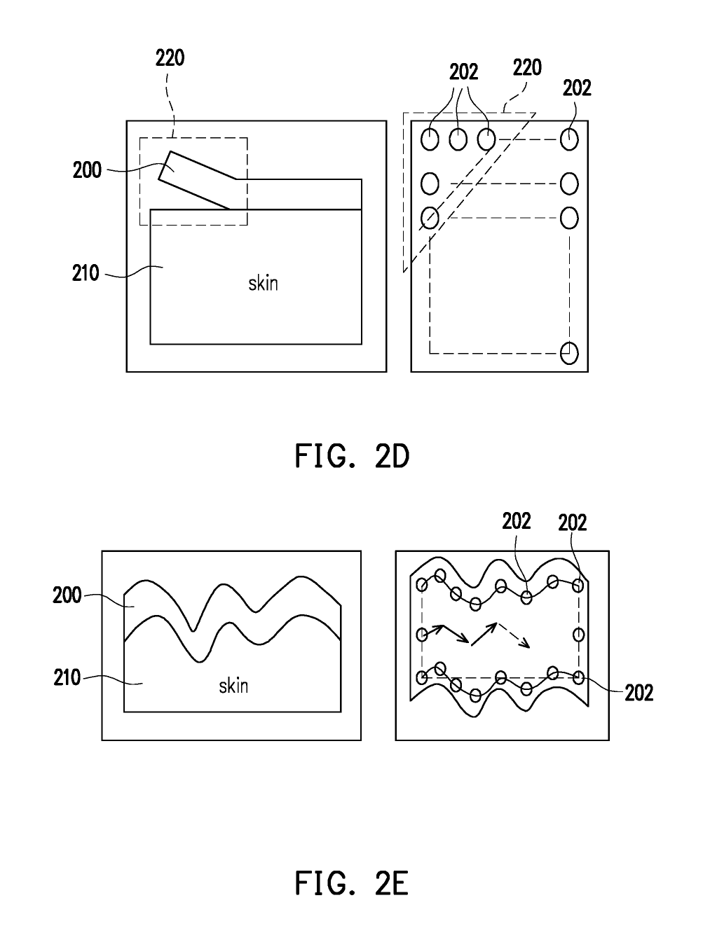

FIG. 2A to FIG. 2C are diagrams illustrating corresponding positional relationships of the physiological signal sensor 110 and the warping sensor 120. A person implementing the embodiment may adjust the correspondence relationship between the physiological signal sensor 110 and the warping sensor 120 according to the requirements and the implementation type of the warping sensor 120. As shown in FIG. 2A, the warping sensor 120 is disposed under the physiological signal sensor 110, and the warping sensor 120 may be implemented as a resistance-type, capacitance-type, photosensitive-type, vibration-type, or electric wave-type sensor. As shown in FIG. 2B, the warping sensor 120 is disposed on the physiological signal sensor 110, and the warping sensor 120 may be implemented as a photosensitive-type, vibration-type, or electric wave-type sensor. As shown in FIG. 2C, the physiological signal sensor 110 is located on the same layer as the warping sensor 120 and the warping sensor 120 is disposed around the physiological signal sensor 110, and the warping sensor 120 may be implemented as a resistance-type, capacitance-type, photosensitive-type, vibration-type, or electric wave-type sensor.

FIG. 2D to FIG. 2E are schematic diagrams illustrating a sensing electrode patch 200 composed of the warping sensor 120 and the physiological signal sensor 110 and an object to be detected (a skin 210). The warping sensor 120 in FIG. 2D to FIG. 2E is an entire surface-type, regional-type, or array-type sensor. In other words, in addition to including the sensing electrode(s) used to sense the physiological signal value, the sensing electrode patch 200 further includes a plurality of evenly distributed sensing points 202 of the warping sensor 120 on the entire sensing electrode patch 200. In FIG. 2D, the sensing electrode patch 200 is mostly closely attached to the object to be detected (the skin 210) and is warped only in a partial region. The left portion of FIG. 2D shows the attachment of the sensing electrode patch 200 and the object to be detected (the skin 210), and a region 220 is where a warping condition of mutual detachment between the sensing electrode patch 200 and the object to be detected (the skin 210) is found. The right portion of FIG. 2D is a schematic diagram illustrating the distribution of the plurality of sensing points 202 on the sensing electrode patch 200. A portion of the sensing electrode patch 200 in the region 220 cannot detect the physiological signal value because it is not closely attached to the skin 210. In addition, the sensing points 202 located in the region 220 also have different sensing signals compared to the other sensing points 202 located outside the region 220 because they are not in contact with the skin 210. The left portion of FIG. 2E shows the attachment of the sensing electrode patch 200 to the object to be detected (the skin 210), and the sensing electrode patch 200 and the object to be detected (the skin 210) are closely attached to each other and are both significantly deformed. Therefore, a part of the sensing points 202 located in the middle of the sensing electrode patch 200 may not be closely attached to the object to be detected (the skin 210), which causes the warping condition of mutual detachment between the sensing electrode patch 200 and the object to be detected (the skin 210).

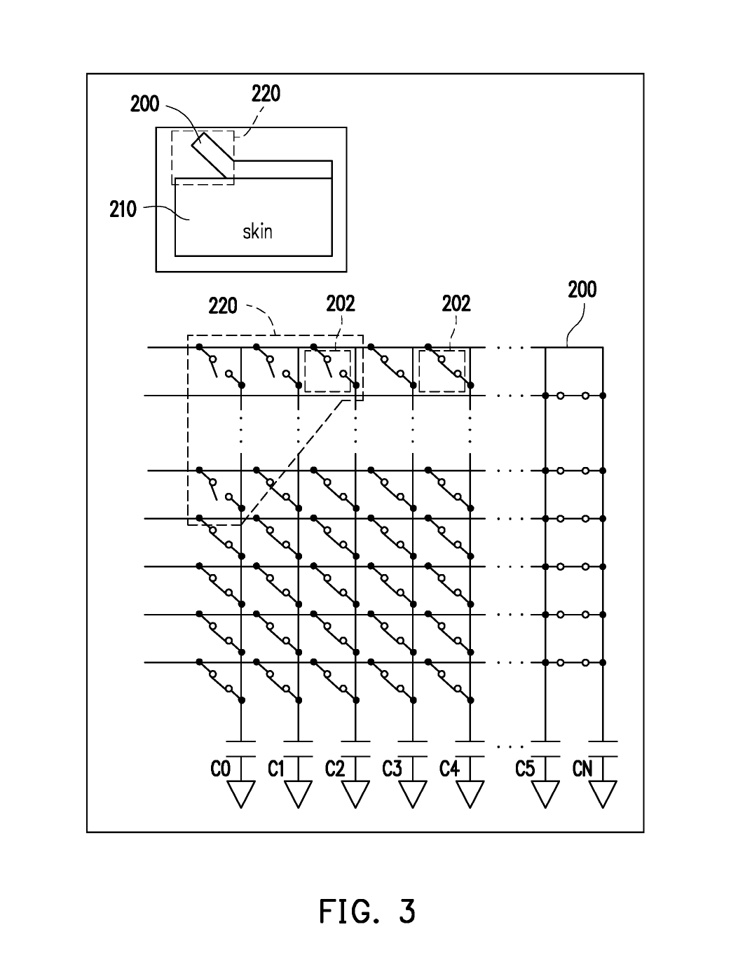

FIG. 3 is a schematic diagram illustrating an implementation of the warping sensor of the sensing electrode patch 200 in FIG. 2D. The sensing electrode patch 200 in FIG. 3 includes the plurality of sensing points 202 of the warping sensor 120. In the embodiment, the warping sensor in the sensing electrode patch 200 is implemented as a capacitance-type sensor. In other words, each of the sensing points 202 in the embodiment is implemented as a switch. Each of the switches in a column is connected to corresponding capacitors C0 to CN, wherein N is a positive integer. When the sensing point 202 implemented as a switch is in contact with the object to be detected (the skin 210), the sensing point 202 is turned on. Conversely, when the sensing point 202 is not in contact with the object to be detected (the skin 210), the sensing point 202 is turned off. Accordingly, when the warping condition above occurs (for example, when none of the sensing points 202 in the region 220 is in contact with the skin 210), the area percentage of the region 220 in the entire sensing electrode patch 200 can be estimated as the warping value based on the change in the total of the capacitance values in the capacitors C0 to CN. A person implementing the embodiment may also use a photosensitive element, a resistive element, a vibration element, a microwave element, etc. as the switch of the sensing point 202 to thereby implement the warping sensor based on various detection methods.

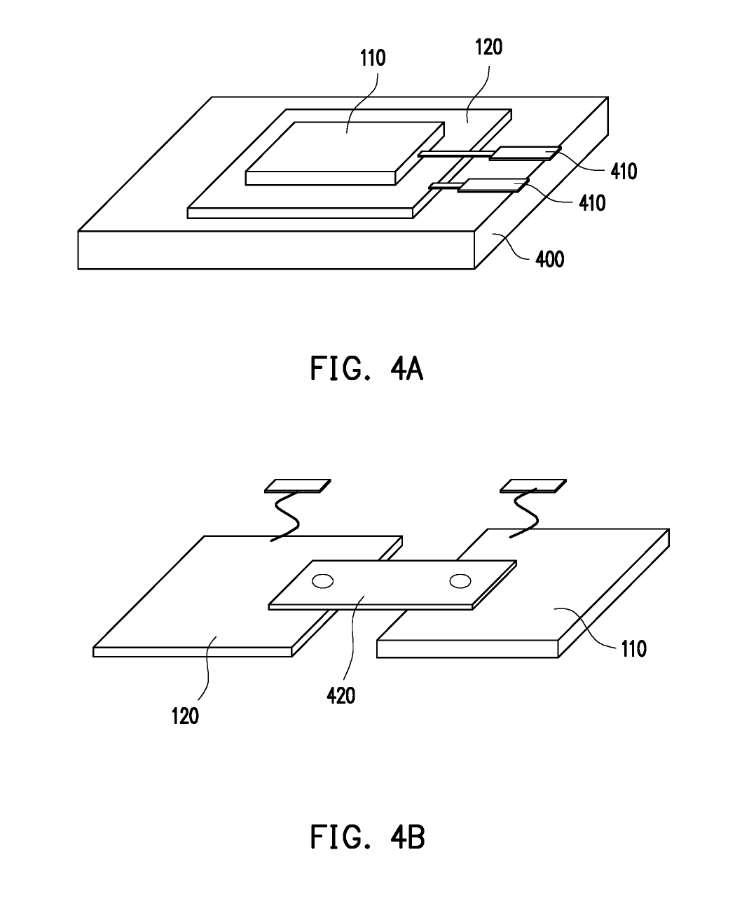

FIG. 4A and FIG. 4B are schematic diagrams illustrating integrated structures of the physiological signal sensor 110 and the warping sensor 120. Referring to FIG. 4A, the warping sensor 120 and the physiological signal sensor 110 may be integrated on one single substrate 400 through a semiconductor manufacturing process, and the pins of the warping sensor 120 and the physiological signal sensor 110 are pulled out to corresponding pads 410. Referring to FIG. 4B, the warping sensor 120 and the physiological signal sensor 110 are produced respectively through different semiconductor manufacturing processes, and the two components are connected to each other by a connection structure 420 (e.g., a conductive paste, an electrode, a screw, or a combination of these components) to integrate the warping sensor 120 and the physiological signal sensor 110 by assembly or pressing.

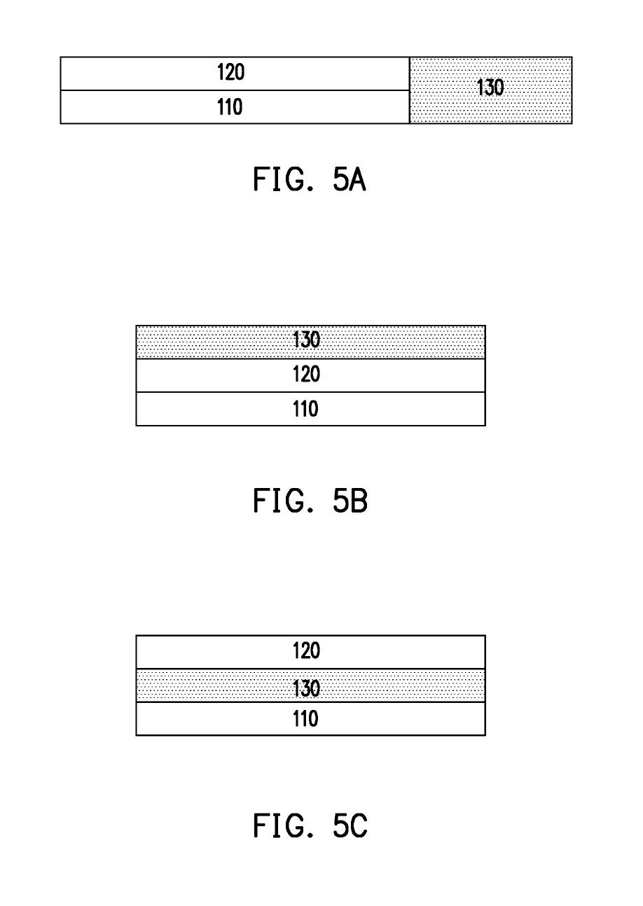

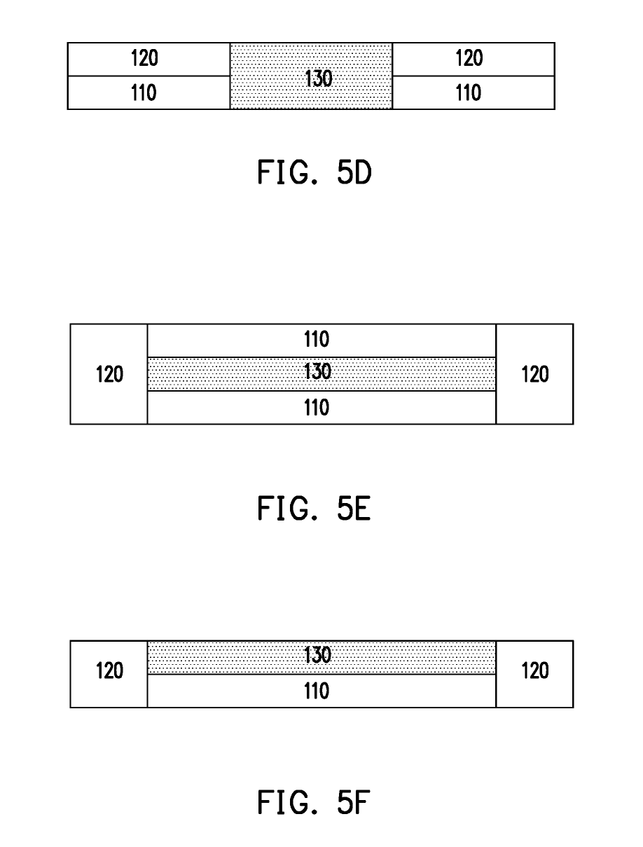

FIG. 5A to FIG. 5F are diagrams illustrating corresponding positional relationships of the physiological signal sensor 110, the warping sensor 120, and the signal processing device 130 in FIG. 1. A person implementing the embodiment may integrate the physiological signal sensor 110, the warping sensor 120, and the signal processing device 130 into the physiological signal correction device 100 according to the requirements. As shown in FIG. 5A, a warping sensor 120 is disposed on the physiological signal sensor 110, and the signal processing device 130 is disposed on the side of the physiological signal sensor 110 and the warping sensor 120. As shown in FIG. 5B, the warping sensor 120 is disposed on the physiological signal sensor 110, and the signal processing device 130 is disposed on the warping sensor 120. As shown in FIG. 5C, the signal processing device 130 is disposed on the physiological signal sensor 110, and the warping sensor 120 is disposed on the signal processing device 130. As shown in FIG. 5D, in addition to the structure of FIG. 5A, the physiological signal sensor 110 and the warping sensor 120 may be further disposed on the other side of the signal processing device 130. As shown in FIG. 5E, the physiological signal sensor 110 is disposed on and under the signal processing device 130, and the warping sensor 120 is disposed on two sides or around the signal processing device 130 and the physiological signal sensor 110, such that the structure of the physiological signal correction device 100 is similar to that in FIG. 2C. As shown in FIG. 5F, the physiological signal sensor 110 is disposed under the signal processing device 130, and the warping sensor 120 is disposed on two sides or around the signal processing device 130 and the physiological signal sensor 110, such that the structure of the physiological signal correction device 100 is similar to that in FIG. 2C.

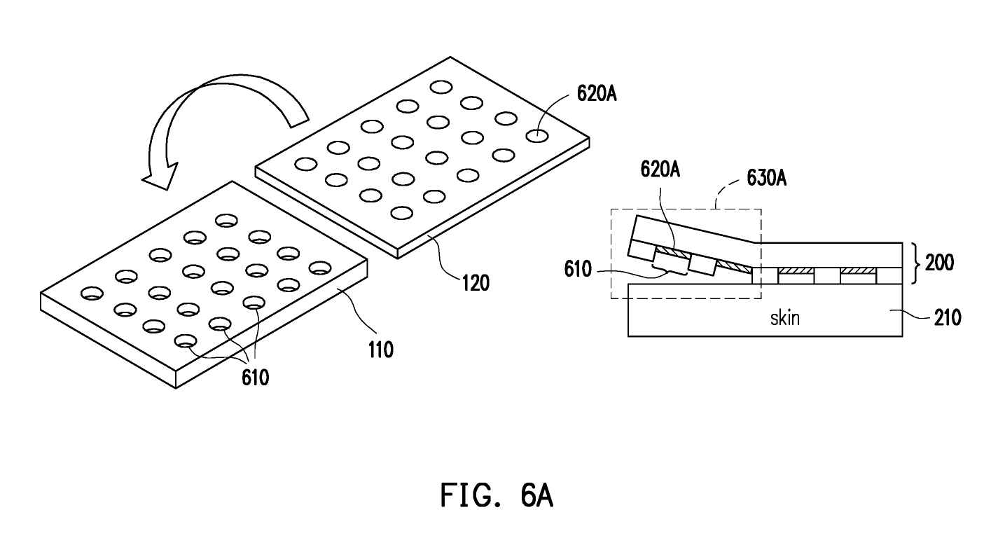

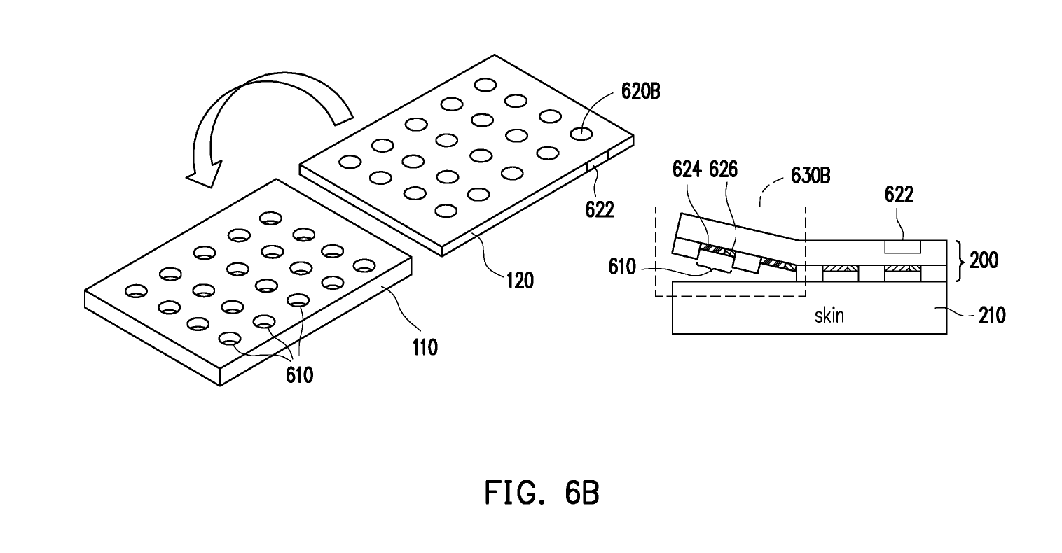

FIG. 6A and FIG. 6B are schematic diagrams illustrating an implementation of the warping sensor 120 as a photosensitive-type sensor. Referring to FIG. 6A, the left portion of FIG. 6A illustrates the physiological signal sensor 110 and the warping sensor 120. The physiological signal sensor 110 includes a plurality of through-holes 610 that allow light to pass through. The plurality of switches in the warping sensor 120 are implemented as a plurality of photosensitive elements 620A. The light sensing surface of the photosensitive element 620A is configured to face the through-hole 610. In the embodiment, each of the through-holes 610 respectively corresponds to one of the photosensitive elements 620A. The photosensitive element 620A may be a photo sensor that generates a corresponding photocurrent according to the amount of light. The right portion of FIG. 6A illustrates the sensing electrode patch 200 composed of the physiological signal sensor 110 and the warping sensor 120 and the object to be detected (the skin 210). When a warping condition occurs in a region 630A, the external light passes through the through-holes 610 from the warped portion, such that the photosensitive elements 620A turn from a non-light-sensing state into a light-sensing state to generate a photocurrent. Thereby, it can be detected that a part (e.g., the region 630A) of the sensing electrode patch 200 is warped, and the area of the region 630A can be detected based on the magnitude of the photocurrent.

Referring to FIG. 6B, the left portion of FIG. 6B also illustrates the physiological signal sensor 110 and the warping sensor 120. The main difference between FIG. 6A and FIG. 6B lies in that, in FIG. 6B, each of the switches of the warping sensor 120 is implemented as a switch device 620B integrating a photosensitive element and a light emitting element (e.g., a light emitting diode (LED)). In other words, in addition to the photosensitive element, the switch in the warping sensor 120 further includes a light emitting element corresponding to each of the photosensitive elements. The light-sensing surface of the switch device 620B is configured to face the through-hole 610. Each of the through-holes 610 respectively corresponds to one of the switch devices 620B. Each photosensitive element and the corresponding light emitting element are disposed in at least one of a plurality of regions of the warping sensor. In addition, an ambient light sensor 622 is further disposed on the rear surface of the warping sensor 120.

The right portion of FIG. 6B illustrates the sensing electrode patch 200 composed of the physiological signal sensor 110 and the warping sensor 120 and the object to be detected (the skin 210). Each region in the sensing electrode patch 200 includes photosensitive elements 624 and light emitting elements 626. The photosensitive element 624 and the light emitting element 626 form the switch device 620B in the left portion of FIG. 6B. When the ambient light sensor 622 on the sensing electrode patch 200 is in a light-sensing state due to sufficient external light, the light emitting element 626 will not actively emit light. At this time, the external light passes through the through-holes 610 from the warped portion (a region 630B), such that the photosensitive elements 624 turn from the non-light-sensing state into the light-sensing state to generate a photocurrent. Thereby, it can be detected that a part (e.g., the region 630B) of the sensing electrode patch 200 is warped. Conversely, when the ambient light sensor 622 on the sensing electrode patch 200 is in a non-light-sensing state due to external light, the light emitting element 626 will actively emit light. At this time, the amount of light sensed by the photosensitive elements 624 at the warped portion (the region 630B) is reduced due to leakage of the light of the light emitting elements 626 (namely, the photocurrent generated by the photosensitive elements 624 is reduced). Thereby, it can be detected that a part (e.g., the region 630B) of the sensing electrode patch 200 is warped. A person implementing the embodiment may also replace the photosensitive element 620A in FIG. 6A with a microwave element, a vibration sensor, a resistance-type sensor, or a capacitance-type sensor. Accordingly, it can be detected based on different detection techniques whether a warping condition occurs, and the area percentage of mutual attachment between the physiological signal sensor 110 and the object to be detected (the skin 210) can be used as the warping value.

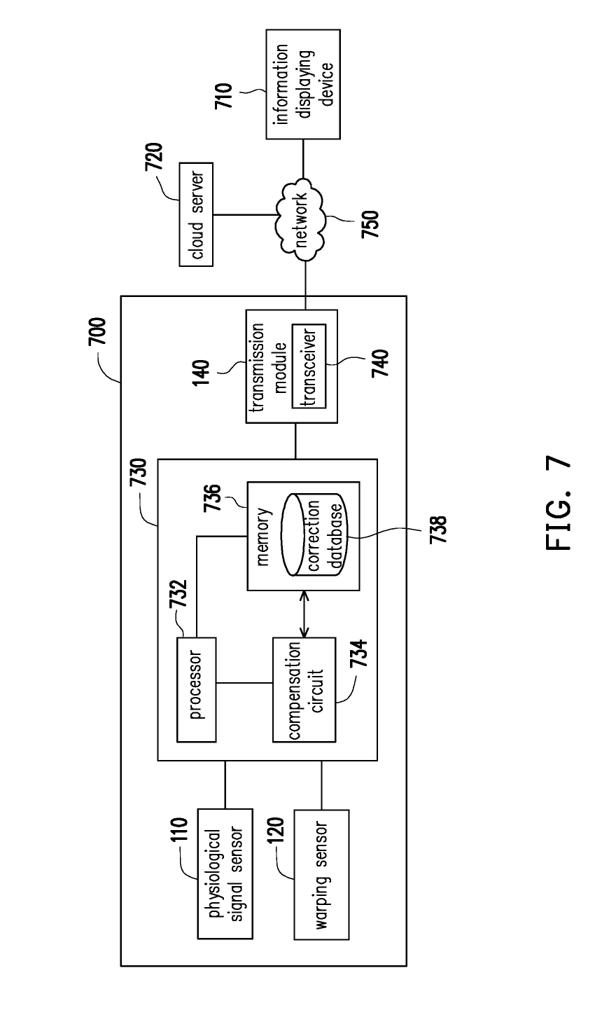

FIG. 7 is a block diagram illustrating a physiological signal correction device 700, an information displaying device 710, and a cloud server 720 according to a second embodiment of the disclosure. The physiological signal correction device 700 includes a physiological signal sensor 110, a warping sensor 120, a signal processing device 730, and a transmission module 140.

The transmission module 140 includes a transceiver 740. After obtaining the corrected physiological signal values, the signal processing device 730 of the physiological signal correction device 700 may integrate the corrected physiological signal values on its own and transmit the physiological signal values to the information displaying device 710 through the transceiver 740 via a network 750 or a relevant transmission protocol (e.g., Bluetooth, WIFI, etc.). Alternatively, the physiological signal correction device 700 may directly transmit the corrected physiological signal values to the information displaying device 710 through the transceiver 740 to have the information displaying device 710 integrate the physiological signal values on its own. The information displaying device 710 may be a smartphone, a tablet computer, a personal computer or server with a screen, etc. and is mainly used to display the wearer's physiological signal values (e.g., the body temperature, the pulse, the heart rate, the respiratory rate, and the dynamic myoelectric current values). The integrated or corrected physiological condition or physiological information (e.g., the wearer's muscular endurance, muscle strength, muscle fatigue, physical condition, exercise cycle, health status, and abnormality alert) may also be displayed by using the information displaying device 710.

The signal processing device 730 and its internal components in the physiological signal correction device 700 in FIG. 7 are described in detail herein. The signal processing device 730 includes a processor 732, a compensation circuit 734, and a memory 736. The compensation circuit 734 is coupled to the processor 732. The memory 736 is coupled to both the processor 732 and the compensation circuit 734. The memory 736 includes a correction database 738.

The correction database 738 at least includes correction signal values corresponding to the warping data generated by the physiological signal sensor 110 and the warping sensor 120. The processor 732 of the embodiment can communicate with the cloud server 720 through the transceiver 740 and can update the contents of the correction database 738 through the cloud server 720 to make the correction of the physiological signal values more accurate.

According to the warping condition provided by the warping sensor 120 (e.g., the area percentage of mutual attachment between the physiological signal sensor 110 and the object to be detected), the compensation circuit 734 queries the correction database 738 to obtain a corresponding correction signal value and provides the correction signal value to the processor 732. The processor 732 adds the correction signal value to the physiological signal value provided by the physiological signal sensor 110 to obtain a corrected physiological signal value. Moreover, the processor 732 in the signal processing device 730 can obtain the corrected physiological signal value corresponding to each time point according to a plurality of time points, perform data calculation on the corrected physiological signal values corresponding to the time points to obtain a plurality of analysis data, and integrate the analysis data and transmit the analysis data to the information displaying device 710 through the transceiver. The information displaying device 710 displays the analysis data on its display screen for viewing by the user. The analysis data above may be presented by using a data link diagram or other graphical data. In some embodiments, the analysis data above may also be uploaded to the cloud server 720 for use in big data analysis and correction of relevant data.

Herein, the contents in the correction database 738 are schematically presented in tables (Table 1: contact area between the sensing electrode(s) and the human body; Table 2: deformation area of the sensing electrode(s)) for reference. A person implementing the embodiment may present the relationship between the warping value and the correction signal value corresponding to the “warping condition” by using more complicated database information.

TABLE 1

| Human body contact area | Compensation/correction | Correction signal |
|---|---|---|
| (area percentage) | level | value |
| 100% | 1 | 0000 |
| 90% | 2 | 0001 |
| 80% | 3 | 0010 |
| 70% | 4 | 0011 |
| 60% | 5 | 0100 |
| 50% | 6 | 0110 |
| 40% | 7 | 0111 |
| 30% | 8 | 1000 |
| 20% | 9 | 1001 |

The warping value transmitted by the warping sensor 120 to the compensation circuit 734 is generally analog information, e.g., a change in the capacitance value (capacitance-type sensor), a change in the photocurrent (photosensitive-type sensor), a change in the resistance value (resistance-type sensor), etc. The compensation/correction levels may be set as different values according to the change values of the analog information above in the actual design. The correction signal values are encoded values of optimal digital resolution set according to the compensation/correction levels. The compensation circuit 734 calculates the area percentage according to the warping value and looks up Table 1 by using the calculated area percentage to thereby obtain the corresponding correction signal value.

TABLE 2

| Deformation area | Compensation/correction | Correction signal |
|---|---|---|
| (area percentage) | level | value |
| 0% | 1 | 0000 |
| 10% | 2 | 0001 |
| 20% | 3 | 0010 |
| 30% | 4 | 0011 |
| 40% | 5 | 0100 |
| 50% | 6 | 0110 |
| 60% | 7 | 0111 |
| 70% | 8 | 1000 |
| 80% | 9 | 1001 |
| 90% | 10 | 1010 |
| 100% | 11 | 1011 |

According to Table 2, the higher the percentage of the deformation area of the physiological signal sensor/sensing electrode(s), the higher the compensation/correction level as well as the correction signal value.

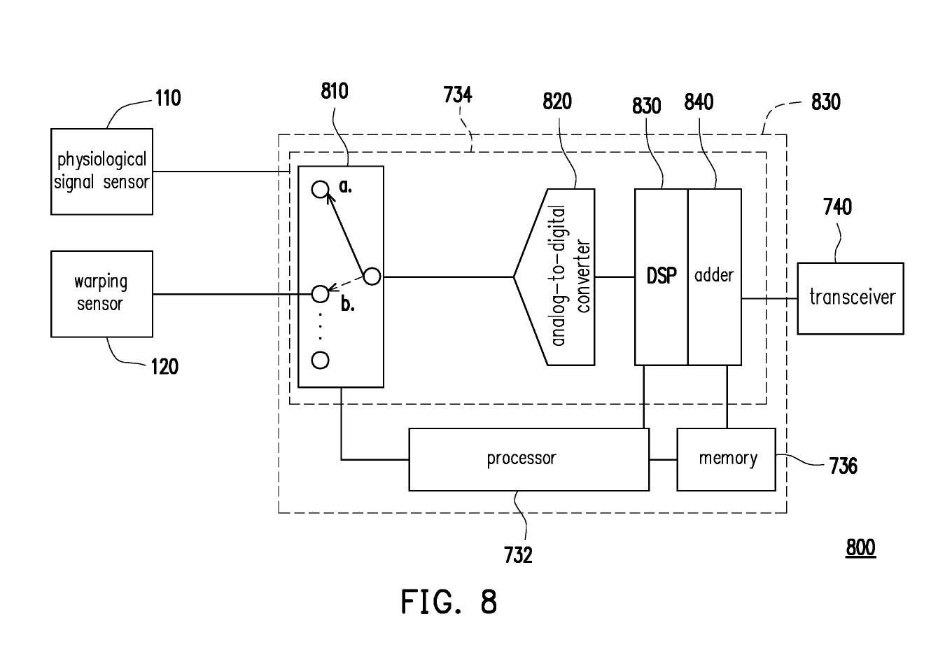

FIG. 8 is a circuit block diagram illustrating a physiological signal correction device 800 according to a third embodiment of the disclosure. In the embodiment, the detailed circuit structure of the compensation circuit 734 is described with reference to FIG. 8. The compensation circuit 734 includes a switcher 810, an analog-to-digital converter 820, a digital signal processor (DSP) 830, and an adder 840. When the sensing electrode patch composed of the warping sensor 120 and the physiological signal sensor 110 is fully attached to the object to be detected, the processor 732 performs an initial operation at time T0 to obtain an initial signal value. The “initial signal value” described in the embodiment may be a compensation value calculated based on a database pre-built on the cloud server 720 of FIG. 7 or parameters (e.g., the user's physiological age/height/weight/blood pressure, environmental parameters such as temperature/humidity/wind force/wind direction/UV radiation) in the correction database 738. A person implementing the embodiment is not limited in terms of the source for obtaining the initial signal value. For convenience of illustration, the initial signal value is set to “0110” in the embodiment. In some embodiments, it is also possible that the processor 732 does not perform the initial operation. At time T1, the processor 732 controls the switcher 810 to obtain a physiological signal value (e.g., the physiological signal value is “0000”) from the physiological signal sensor 110. At time T2, the processor 732 controls the switcher 810 to obtain a warping value from the warping sensor 120, utilizes the analog-to-digital converter 820 and the digital signal processor 830 to digitally encode the warping value, and queries the correction database in the memory 736 by using the warping value to obtain a correction signal value. In an embodiment, if there is no correction signal value and there is an initial signal value, the processor 732 controls the adder 840 to add the initial signal value (“0110”) to the physiological signal value (“0000”) as the physiological signal value. In another embodiment, if there is a correction signal value (e.g., the correction signal value is “1000”) and there is an initial signal value, the processor 732 adds the initial signal value (“0110”) to the physiologic signal value (“0000”) and then adds the correction signal value (“1000”) as the corrected physiological signal value. Afterwards, at time T3, the processor 732 transmits the physiological signal value or the corrected physiological signal value above to the information displaying device through the transceiver 740.

The compensation circuit 734 of FIG. 8 is implemented with one single analog-to-digital converter 820. A person implementing the embodiment may also implement the compensation circuit 734 with two analog-to-digital converters 820. One of the analog-to-digital converters is used to convert the physiological signal value of the physiological signal sensor 110 into a digital form, and the other of the analog-to-digital converters is used to convert the warping value of the warping sensor 120 into a digital form. In this way, the switcher 810 is not required to perform signal switching, and the digital signal processor 830 can process the two data at the same time.

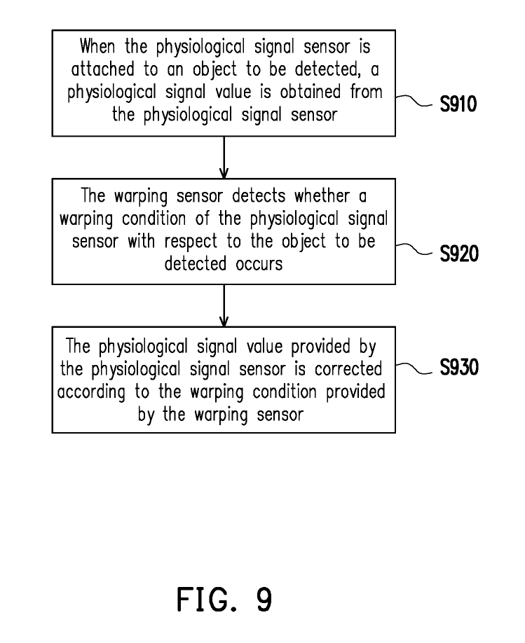

FIG. 9 is a flowchart illustrating a correction method for a physiological signal according to an embodiment of the disclosure. The correction method is adapted for the physiological signal correction device 100, 700, and/or 800 of the embodiments above. Referring to FIG. 9, in step S910, when the physiological signal sensor 110 is attached to an object to be detected, the signal processing device in the physiological signal correction device obtains a physiological signal value from the physiological signal sensor 110. In step S920, the signal processing device detects whether a warping condition of the physiological signal sensor 110 with respect to the object to be detected occurs through the warping sensor 120. The warping condition is caused by a distance between the sensing electrode(s) in a part of the physiological signal sensor 110 and the object to be detected. In step S930, the signal processing device corrects the physiological signal value provided by the physiological signal sensor 110 according to the warping condition provided by the warping sensor 120. Reference may be made to the embodiments above for implementation details of the steps above.

In summary of the above, the physiological signal correction device and the wearable device described in the embodiments of the disclosure utilize the warping sensor disposed on the physiological signal sensor to detect the warping condition between the sensing electrode(s) in the physiological signal sensor and the object to be detected (e.g., the user's skin) and correct the physiological signal according to the warping condition. In other words, in the embodiments of the disclosure, one or multiple types of warping sensors are disposed on the physiological signal sensor (e.g., a sensing electrode patch) to detect and feed back the area percentage value of mutual detachment between the sensing electrode(s) and the object to be detected, and the correction database is queried by using the area percentage value to compensate or correct the missing part of the physiological signal. Thereby, the physiological signal detected in the embodiments of the disclosure can exhibit high accuracy through correction.

Although the embodiments are already disclosed as above, these embodiments should not be construed as limitations on the scope of the disclosure. It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A physiological signal correction device comprising:
a physiological signal sensor comprising at least one sensing electrode, the physiological signal sensor being attached to an object to be detected to obtain a physiological signal value from the sensing electrode;
a warping sensor disposed on the physiological signal sensor, the warping sensor detecting whether a warping condition of the physiological signal sensor with respect to the object to be detected occurs; and
a signal processing device coupled to the physiological signal sensor and the warping sensor,
wherein the signal processing device corrects the physiological signal value provided by the physiological signal sensor according to the warping condition provided by the warping sensor, wherein the warping condition is caused by a distance between a part of the sensing electrode and the object to be detected or a change in a contact area between a part of the sensing electrode and the object to be detected.

2. The physiological signal correction device according to claim 1, wherein a warping value of the warping condition is expressed in an area percentage of mutual attachment and detachment between the physiological signal sensor and the object to be detected, or
the warping value of the warping condition is expressed in an area percentage of an area where the physiological signal sensor is attached to the object to be detected and is deformed.

3. The physiological signal correction device according to claim 1, wherein the signal processing device comprises:
a processor;
a compensation circuit coupled to the processor; and
a memory comprising a correction database,
wherein the compensation circuit queries the correction database according to the warping condition provided by the warping sensor to obtain a correction signal value and provides the correction signal value to the processor, and
the processor adds the correction signal value to the physiological signal value provided by the physiological signal sensor to obtain a corrected physiological signal value.

4. The physiological signal correction device according to claim 3, further comprising:
a transmission module coupled to the signal processing device, wherein a transceiver of the transmission module communicates with an information displaying device,
wherein the signal processing device integrates the corrected physiological signal value and transmits the corrected physiological signal value to the information displaying device, and the information displaying device displays physiological information corresponding to the object to be detected according to the corrected physiological signal value.

5. The physiological signal correction device according to claim 4, wherein the signal processing device obtains an initial signal value from the correction database or a cloud server when the physiological signal sensor and the warping sensor are fully attached to the object to be detected, and after obtaining the initial signal value, the signal processing device obtains the physiological signal value from the sensing electrode of the physiological signal sensor and obtains a warping value corresponding to the warping condition from the warping sensor, and
when the warping value is obtained, the signal processing device queries the correction database according to the warping value to obtain the correction signal value and adds the initial signal value and the correction signal value to the physiological signal value as the corrected physiological signal value.

6. The physiological signal correction device according to claim 5, wherein the signal processing device obtains the corrected physiological signal value corresponding to each time point according to a plurality of time points, performs data calculation on the corrected physiological signal value corresponding to each time point to obtain a plurality of analysis data, and integrates the analysis data and transmits the analysis data to the information displaying device through the transceiver.

7. The physiological signal correction device according to claim 1, wherein the physiological signal sensor comprises at least one through-hole, and the warping sensor is disposed to correspond to the through-hole.

8. The physiological signal correction device according to claim 1, wherein types of the warping sensor comprise a photosensitive-type sensor, a vibration-type sensor, a resistance-type sensor, a capacitance-type sensor, a microwave-type sensor, or a combination thereof.

9. The physiological signal correction device according to claim 8, wherein when the warping sensor is the photosensitive-type sensor, the warping sensor comprises a plurality of photosensitive elements,
wherein detection of whether the warping condition occurs is determined by whether a part of the photosensitive elements sense light.

10. The physiological signal correction device according to claim 9, wherein the warping sensor further comprises a light emitting element corresponding to each of the photosensitive elements,
wherein each of the photosensitive elements and the corresponding light emitting element are disposed in at least one of a plurality of regions of the warping sensor.

11. A correction method for a physiological signal adapted for a physiological signal correction device comprising a physiological signal sensor and a warping sensor, wherein the warping sensor is disposed on the physiological signal sensor, the correction method comprising:
obtaining a physiological signal value from the physiological signal sensor when the physiological signal sensor is attached to an object to be detected;
detecting, by the warping sensor, whether a warping condition of the physiological signal sensor with respect to the object to be detected occurs, wherein the warping condition is caused by a distance between a part of the physiological signal sensor and the object to be detected or a change in a contact area between a part of at least one sensing electrode and the object to be detected; and
correcting the physiological signal value provided by the physiological signal sensor according to the warping condition provided by the warping sensor.

12. The correction method according to claim 11, wherein a warping value of the warping condition is expressed in an area percentage of mutual attachment and detachment between the physiological signal sensor and the object to be detected, or
the warping value of the warping condition is expressed in an area percentage of an area where the physiological signal sensor is attached to the object to be detected and is deformed.

13. The correction method according to claim 11, wherein the physiological signal value provided by the physiological signal sensor according to the warping condition provided by the warping sensor comprises the following step:
adding a correction signal value to the physiological signal value provided by the physiological signal sensor to obtain a corrected physiological signal value.

14. The correction method according to claim 13, further comprising a step below:
obtaining an initial signal value from a correction database or a cloud server when the physiological signal sensor and the warping sensor are fully attached to the object to be detected,
wherein the step of correcting the physiological signal value provided by the physiological signal sensor according to the warping condition provided by the warping sensor comprises steps below:
after the initial signal value is obtained, obtaining the physiological signal value from the physiological signal sensor and obtaining a warping value corresponding to the warping condition from the warping sensor; and
when the warping value is obtained, querying the correction database according to the warping value to obtain the correction signal value, and adding the initial signal value and the correction signal value to the physiological signal value as the corrected physiological signal value.

15. The correction method according to claim 11, further comprising steps below:
obtaining the corrected physiological signal value corresponding to each time point according to a plurality of time points;
performing data calculation on the corrected physiological signal value corresponding to each time point to obtain a plurality of analysis data; and
integrating the analysis data and transmitting the analysis data to an information displaying device.

16. A wearable device with a correction function, comprising:
a physiological signal sensor comprising at least one sensing electrode, the physiological signal sensor being attached to an object to be detected to obtain a physiological signal value from the sensing electrode;
a warping sensor disposed on the physiological signal sensor, the warping sensor detecting whether a warping condition of the physiological signal sensor with respect to the object to be detected occurs; and
a signal processing device coupled to the physiological signal sensor and the warping sensor,
wherein the signal processing device corrects the physiological signal value provided by the physiological signal sensor according to the warping condition provided by the warping sensor, wherein the warping condition is caused by a distance between a part of the sensing electrode and the object to be detected or a change in a contact area between a part of the sensing electrode and the object to be detected.

17. The wearable device according to claim 16, wherein a warping value of the warping condition is expressed in an area percentage of mutual attachment and detachment between the physiological signal sensor and the object to be detected, or
the warping value of the warping condition is expressed in an area percentage of an area where the physiological signal sensor is attached to the object to be detected and is deformed.

18. The wearable device according to claim 16, wherein the signal processing device comprises:
a processor;
a compensation circuit coupled to the processor; and
a memory comprising a correction database,
wherein the compensation circuit queries the correction database according to the warping condition provided by the warping sensor to obtain a correction signal value and provides the correction signal value to the processor, and
the processor adds the correction signal value to the physiological signal value provided by the physiological signal sensor to obtain a corrected physiological signal value.

19. The wearable device according to claim 18, further comprising:
a transmission module coupled to the signal processing device, wherein a transceiver of the transmission module communicates with an information displaying device,
wherein the signal processing device integrates the corrected physiological signal value and transmits the corrected physiological signal value to the information displaying device, and the information displaying device displays physiological information corresponding to the object to be detected according to the corrected physiological signal value.

20. The wearable device according to claim 19, wherein the signal processing device obtains an initial signal value from the correction database or a cloud server when the physiological signal sensor is fully attached to the object to be detected, and after obtaining the initial signal value, the signal processing device obtains the physiological signal value from the sensing electrode and obtains a warping value corresponding to the warping condition from the warping sensor, and
when the warping value is obtained, the signal processing device queries the correction database according to the warping value to obtain the correction signal value and adds the initial signal value and the correction signal value to the physiological signal value as the corrected physiological signal value.
